(19) United States
(12) Patent Application Publication
(10) Pub. No.: US 2021/0259579 A1
(43) Pub. Date: Aug. 26, 2021

(54) DEVICE FOR TREATING IDIOPATHIC TOE WALKING

(71) Applicant: Chapman University, Orange, CA (US)

(72) Inventors: Marybeth Grant-Beuttler, Corona del Mar, CA (US); Rahul Soangra, Foothill Ranch, CA (US)

(21) Appl. No.: 17/181,238

(22) Filed: Feb. 22, 2021

Related U.S. Application Data
(60) Provisional application No. 62/979,806, filed on Feb. 21, 2020.

(51) Int. Cl. A61B 5/103; A61B 5/11; A61B 5/00

(52) U.S. Cl. CPC A61B 5/1038; A61B 5/112; A61B 2562/0219; A61B 2503/06; A61B 5/6807

(57) ABSTRACT

A wearable system for treating a child patient with Idiopathic Toe Walking (“ITW”) is disclosed herein. A disclosed system includes a wearable shoe insole, a first pressure sensor located at a heel region of the shoe insole, a second pressure sensor located at a front region of the shoe insole, and a vibration actuator included within the shoe insole. The disclosed system may also include an inertial measurement unit. The disclosed system further includes a processor that receives data from the pressure sensors and/or the inertial measurement unit. The processor determines a gait pattern of the child patient based on the received data. Indicative of determining the gait pattern is a toe-to-toe gait pattern, the processor causes the vibration actuator to provide haptic feedback to the child patient for correcting their tow walking gait.

### PRIORITY CLAIM

This application claims priority to and the benefit as a non-provisional application of U.S. Provisional Patent Application No. 62/979,806, filed Feb. 21, 2020, the entire content of which is hereby incorporated by reference and relied upon.

### BACKGROUND

By six years of age, approximately one out of every 20 children will demonstrate Idiopathic Toe Walking (“ITW”). Children diagnosed with ITW (“cITW”) demonstrate poorer standing balance and decreased gross motor skills. Additionally, children with ITW demonstrate toe-to-toe contact during gait with infrequent or no contact of the heel with a surface. Current interventions focus on lengthening the calf muscles or using orthotics to restrict movement onto the toes. However, these current known interventions demonstrate limited long-term success for the children.

Parents report that many cITW demonstrate a heel strike with verbal reminders. Despite these reminders, toe-to-toe stepping returns within a few steps or minutes. Continual verbal reminders from parents can frustrate a child. In public, these reminders can draw unwanted attention to the child in front of peers. It is unfeasible for parents to remind children throughout the day. Secondary to limited correction, the cITW rehearse toe-walking more frequently than heel-strike gait.

### SUMMARY

Idiopathic Toe Walking is an abnormal pattern of toe-to-toe contact during gait and is estimated to occur in 5 to 24% of children. Intervention for children with Idiopathic Toe Walking (“ITW”) has demonstrated no long-term success in increasing limited muscle tendon unit length at the ankle or modification of the toe-toe gait pattern. ITW limits balance skills and decreases physical activity in children and adults. ITW even often causes children to expend more energy resulting in fatigue and pain during walking. Assessment and intervention to address modifying the gait pattern is impaired by two issues: (i) a limited ability to monitor a frequency of Toe Walking (“TW”) gait pattern in a child's natural environment and, (ii) limitations in time spent in therapy practicing a desired, normal heel-toe gait pattern. Disclosed herein is a system configured to monitor and intervene with TW. The example system is wearable by the child in a shoe, enabling near constant monitoring and correction at home in the child's natural environment. The example system disclosed herein includes a wearable sensor (e.g., accelerometers and/or gyroscopes) that monitors the use of a toe-to-toe gait pattern and physical activity over multiple days. The example system provides a reminder through haptic feedback (e.g., vibro-tactile) after detecting a child is returning to a toe-to-toe gait instead of a desired heel-to-toe gait. The vibrational reminder is configured to cause the child to concentrate on correcting the toe-to-toe gait by walking with a heel-to-toe gait. The placement of the sensor and the vibration actuator in the shoe provides inconspicuous treatment without raising embarrassing attention to the wearer. The example system may be placed in a shoe for multiple days or weeks to provide extended training as needed to increase the time spent rehearsing a heel-to-toe gait pattern.

The disclosed wearable sensor has the potential to recast the clinical approach to monitor and intervene with children who have TW gait pattern. The disclosed system including the wearable sensor decreases the financial and emotional stress for the children and their families by eliminating the need for repeated bouts of long-term (in-clinic) intervention.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE DRAWINGS

FIG. 1 is a diagram illustrative of a gait for a child without ITW.

FIG. 2 is a diagram illustrative of a postural control strategy (e.g., an ankle strategy) for a child without ITW.

FIG. 3 is a diagram of an ICF model that defines a relationship between body structure and function, activity, and participation in individuals with disabilities.

FIG. 4 is a diagram of an example system to correct or treat ITW, according to an example embodiment of the present disclosure.

FIGS. 5 and 6 are diagrams of a shoe insole of the system of FIG. 4, according to example embodiments of the present disclosure.

FIG. 7 shows the system of FIGS. 4 to 6 connected to a representation of a child's foot via the insole, according to an example embodiment of the present disclosure.

FIG. 8 is a flow diagram of an example procedure for detecting a child patient has a toe-to-toe gait, according to an example embodiment of the present disclosure.

FIG. 9 shows a heel-to-toe gait patent and a toe-to-toe gait pattern, according to an example embodiment of the present disclosure.

FIG. 10 is an example routine that may be performed by a processor to perform a walking classification using the one or more detected gait event(s), according to an example embodiment of the present disclosure.

### DETAILED DESCRIPTION

The example system disclosed herein is configured to provide intervention or treatment for children diagnosed with Idiopathic Toe Walking (“ITW”). The example system includes one or more pressure and/or inertial sensors for detecting a gait pattern. The example system also includes a processor configured to detect when a child deviates from a desired heel-to-toe gait pattern. If a deviation is detected, the example system is configured to provide haptic feedback (e.g., biofeedback) via a vibration actuator. The haptic feedback is configured to get a child's attention causing them to focus on walking with a heel-to-toe gait. Overtime, the haptic feedback causes a behavioral change in children with ITW causing them to walk with a normal heel-to-toe gait without having to concentrate on their walking. The example system is configured for children across the spectrum of severity for ITW.

In some embodiments, the example system is configured as an insole of a shoe. A processor included within the insole is configured to track a history of a child's gait pattern. A clinician may view the gait pattern to determine how quickly a child's ITW is decreasing in severity. A lack of change may be indicative that more intense treatment may be needed to address the child's ITW condition. Further, the history data may be used in clinical studies for further understanding ITW.

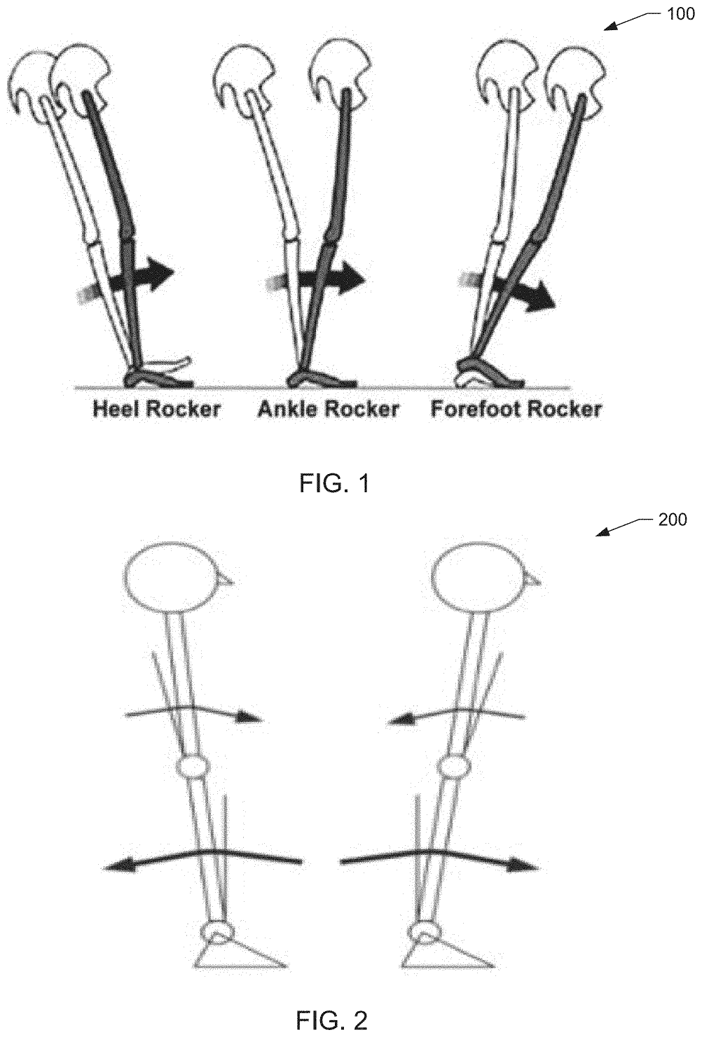

Most children begin walking around the first year of life. During early walking, toddlers demonstrate large variability between foot flat and heel strike contact. Over the next six to twelve months of walking, children increase the consistency of heel strike as they learn to use the weight bearing surface of their heels. FIG. 1 is a diagram illustrative of a typical gait 100 for a child. As shown, the normal gait 100 includes three sequential gait rockers. A first gait rocker includes a heel strike to full foot contact (heel rocker). A second rocker includes movement of a child's leg over the flat foot (ankle rocker). A third rocker includes the flat foot pushing off on the toes (forefoot rocker). Parent's report initial observations of toe walking in children around two years of age.

Children develop these gait rockers to decrease the energy cost of walking. During these early months of walking, children are also developing a coordinated motor response to control posture, referred to as an ankle strategy. FIG. 2 is a diagram that is illustrative of a child's postural control strategy, also known as an ankle strategy 200. As illustrated, the ankle strategy 200 defines a weight shift over a child's foot. The ankle strategy 200 is used to maintain balance on firm surfaces and relies heavily on somatosensory input from the bottom of the foot. Both the gait rockers (shown in FIG. 1) and the ankle strategy 200 use contact with the floor to control movement of the body.

Studies have shown that children with ITW never develop the gait rockers shown in FIG. 1 and the ankle strategy 200 shown in FIG. 2. Instead of using their heels, children with ITW stand and walk primarily on their toes. However, standing on one's toes limits foot contact with a surface and decreases somatosensory input. In addition, standing on the toes places the muscles in the front and back of the lower leg in either an over-lengthened or over-shortened position. Both over lengthening or shortening a muscle decreases the ability of the muscle to produce force. It is theorized that standing on one's toes results in an inability to develop controlled weight shift (e.g., a Center of Pressure (“COP”) movement from heel-to-toe).

Reports on the incidence of ITW at six years of age are limited and vary wildly with reports as high as 22% and as low as 4.9% of all children. A conservative estimate of 5% suggests that over 3.6 million children in the US could currently be diagnosed with ITW. Even this conservative estimate makes ITW more than 4.5 times more prevalent than cerebral palsy.

The progression of ITW is highly debatable. Multiple studies have documented essentially no change or some improvement in gait with age, but suggest that children with ITW never develop “typical” gait. A recent study suggested 79% of children with ITW “spontaneously” develop a typical gait by 10 years of age. This study demonstrates flaws in calculations of their “spontaneous recovered” group, and in addition, fail to confirm recovery of a typical gait pattern via observation. Most studies focus solely on the presence of heel strike during gait with lack of attention to the gait rockers or other functional limitations which may persist. Based on the majority of the evidence, structural and functional limit

Most known ITW interventions and treatments focus on correcting heel strike using two approaches: 1) lengthen the calf muscle (gastrocnemius and soleus, or triceps surae), and 2) restricting movement of the foot into a toe down position (plantarflexion). Intervention and treatments to lengthen the calf muscles can include serial casting the ankle into a maximum toes up (dorsiflexion) position, injecting Botox® into the calf muscles to temporarily paralyze muscles from pulling the foot into a toes down (plantar flexed) position, or surgical lengthening calf muscles to limit pulling the foot into a toes down position. All of these interventions or treatments to lengthen the calf muscle have demonstrated improvements in passive movement into a toes up position (dorsiflexion). Additionally, some studies report an immediate decrease in weight bearing on the toes. However, evidence of long-term maintenance of muscle lengthening is limited. While shortening of the calf muscles is frequently observed in ITW, intervention to lengthen this muscle assumes this is the cause of ITW. Based on the fact some studies report a higher incidence of shortened calf muscles in older children and others note shortening returns following intervention, this shortening may be a response of the muscle to produce power in a shortened position (standing on the toes). If shortening of the calf muscle is a result of ITW, intervention and treatment should instead focus on retraining the muscle to work in a lengthened position (dorsiflexion).

A second approach to ITW intervention and treatment uses orthotics or shoe inserts to restrict toes down movement at the ankle and foot (plantarflexion). While ankle-foot orthoses have an immediate effect of blocking standing on the toes, they negatively affect development of the gait rockers. Long-term outcomes suggest children frequently return to toe walking when the device is removed. In addition, foot orthotics must be worn for prolonged periods (over 2 to 4 years) with no evidence to suggest remediation of a normal gait pattern. While using ankle-foot and foot orthotics appear to immediately reduce toe touch and can be worn throughout the day, blocking the use of the foot and ankle does not “re-teach” the gait rockers and explains why return of the ITW pattern is frequently observed when these orthoses are removed. The system disclosed herein provides continuous, intelligent feedback to increase active use of the gait rockers to retrain the desired gait pattern and increase the likelihood that these patterns will be maintained following intervention.

A few known studies have applied motor “relearning” interventions with ITW to increase heel strike frequency. Some of these known studies provided augmented feedback through auditory and through visual feedback. All of these studies reported increased heel strike frequency over multiple days and weeks of training. While these studies documented success in increasing heel strike, they were not able to provide these interventions in all environments or for extended periods of time. The example system disclosed herein provides motor relearning throughout the day and in the natural environment has the potential to maximize rehearsal time and modify a well-engrained movement pattern.

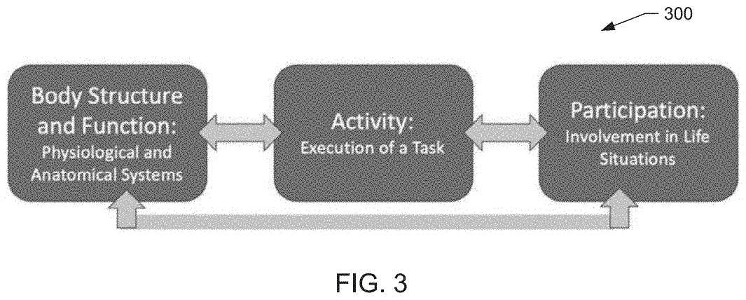

Over the last two years, specific gait and balance limitations in children with ITW were documented in development of the system disclosed herein. Limitations were documented across all levels of an International Classification of Functioning (“ICF”) model. FIG. 3 is a diagram of an ICF model 300 that defines a relationship between body structure and function, activity, and participation in individuals with disabilities. For development of the system disclosed herein, the ICF model was created using a cohort of 32 children ages 4-14 (mean age=8.9±2.9 years, 59% male). Performance level outcomes suggest children with ITW demonstrate decreased steps per day, an increase in falls, and decreased endurance. Activity level measurements suggest lower gross motor development specifically in tasks challenging postural control. Body structure and function issues include complaints of pain, limited ankle range of motion (e.g., ankle strategy 200), longer muscle latency to postural control challenges, over reliance on vision with decreased reliance on somatosensory information during stance, and poor foot position (pronation). The example system disclosed herein is configured to address limitations in the development of the gait rockers and postural control that contribute to limited ability to execute daily tasks.

While the system is disclosed herein as providing treatment for children with ITW, it should be appreciated the system may be used for other conditions. For example, the system may be used to treat cerebral palsy, spinal cord injuries, muscular dystrophies, autism, children with learning disabilities, and/or children with developmental coordination disorder and neuromuscular disorders. Further, while the system is disclosed as providing treatment for children, in other embodiments the system may be used for adults who toe-walk or adults that have disorders related to walking or gait issues generally.

### Example System to Treat ITW

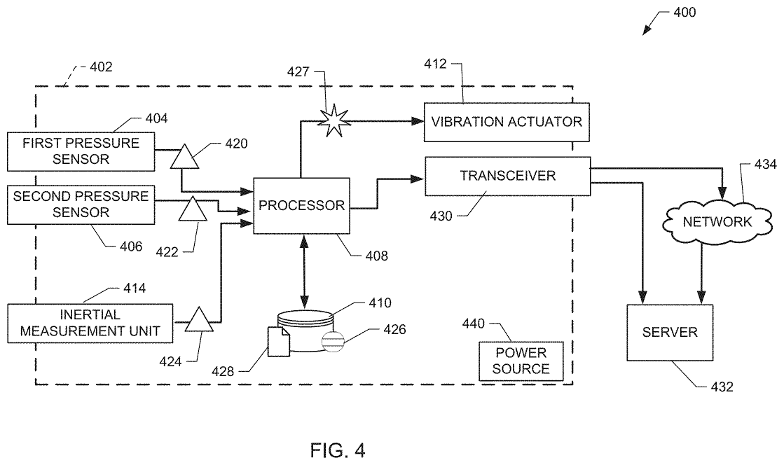

FIG. 4 is a diagram of an example system 400 to correct or treat ITW, according to an example embodiment of the present disclosure. The example system 400 is configured to detect the absence of the gait rockers shown in FIG. 1 and the ankle strategy 200 shown in FIG. 2. The example system 400 uses one or more pressure sensors and/or inertial measurement units to determine whether a child patient is walking with a toe-to-toe gait or a heel-to-toe gait. The sensors are positioned to detect whether the gait rockers shown in FIG. 1 and the ankle strategy 200 shown in FIG. 2 are occurring during a child's walking and/or running.

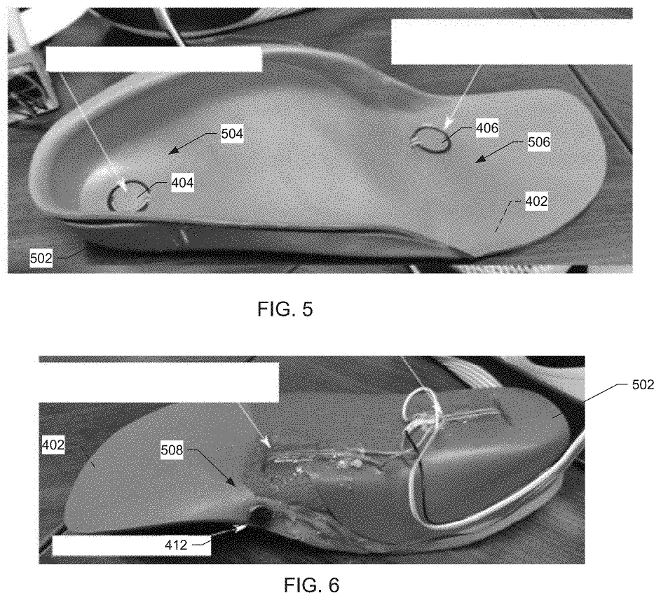

The example system 400 of FIG. 4 is self-contained within a shoe insole 402. FIGS. 5 and 6 are diagrams of the shoe insole 402, according to example embodiments of the present disclosure. The example shoe insole 402 is configured to fit within a shoe of a child. In some embodiments, the insole 402 may be customized based on a shape and/or size of a child's foot. The insole 402 may be made from any rubber, plastic, form, or combinations thereof to provide shaped-foot support.

The insole 402 may include an arch support 502. As shown in FIGS. 5 and 6, the arch support 502 is located on a bottom side of the insole 402. In some embodiments, the arch support 502 is positioned from a rear of the insole 402 to a mid-positon of the insole 402 just before a region that contacts a patient's metatarsals. The arch support 502 may be made from a same material as the insole 402. Alternatively, the arch support 502 may include a hard plastic while the insole 402 includes a softer material.

Returning to FIG. 4, the example system 400 includes a first pressure sensor 404, a second pressure sensor 406, a processor 408, a memory device 410, and a vibration actuator 412. In some embodiments, the system 400 may include additional pressure sensors or fewer pressure sensors. Further, in some embodiments, the system 400 may include an inertial measurement unit 414.

As shown in FIG. 5 the first pressure sensor 404 is located at a heel region 504 of the insole 402. The first pressure sensor 404 is configured to detect contact with a patient's heel during walking. The second pressure sensor 406 is located at a front region 506 of the insole 402. The front region 506 is a region that is aligned with a first metatarsal, a second metatarsal, a third metatarsal, a first phalange, a second phalange, and/or a third phalange of the child patient. The second pressure sensor 406 is configured to detect contact with a patient's toes during walking.

The pressure sensors 404 and 406 may include force sensing resistors (“FSRs”) that are configured to detect forces between 0 pounds-per-square inch (“lb/in²) and 4 lb/in². In some embodiments, the pressure sensors 404 and 406 may include FSRs manufactured by Adafruit®. While FSRs are disclosed herein, it should be appreciated that the pressure sensors 404 and 406 may include any sensor to detect force from a foot strike including capacitive sensors, piezoresistive sensors, etc.

Returning to FIG. 4, the first pressure sensor 404 is configured to transmit first pressure data 420 and the second pressure sensor 406 is configured to transmit second pressure data 422. The sensors 404 and 406 may be configured to transmit a near-continuous stream of pressure data. In some examples, sensors 404 and 406 may be configured to transmit the data 420 and 422 at a sample period may be every 500 milliseconds, 250 milliseconds, 100 milliseconds, etc. In some instances, the processor 408 transmits a trigger signal that causes the pressure sensors 404 and 406 to transmit a current measured pressure.

The first and second pressure data 420 and 422 may include an analog waveform or a digital signal. The data 420 and 422 is indicative of a measured pressure. The data 420 and 422 is configured to indicate a pressure between 0 lb/in² and 4 lb/in².

The example inertial measurement unit 414 may be included within the system 400 when acceleration or rotational acceleration data is used for determining a gate type of a user. The inertial measurement unit 414 may include at least one accelerometer and/or at least one inertial sensor or gyroscope. The accelerometer may foot movement foot-backward, side-to-side or up-down. The gyroscope measures foot movement along angular axes including a roll axis, a pitch axis, and a yaw axis. The inertial measurement unit 414 may include the MPU-9250 TDK InvenSense sold by Digi-Key Electronics®.

The inertial measurement unit 414 is configured to transmit data 424 to the processor 408. The inertial measurement unit 414 may transmit data 424 in response to a trigger signal from the processor 408, at periodic sampling intervals, and/or at a continuous data stream. The data 424 may be indicative of an acceleration and/or an angular acceleration.

The example vibration actuator 412 is configured to provide haptic feedback to a child patient. The vibration actuator 412 may include the 1597-1244-ND from Seeed Technology Co.® or any other actuator that produces mechanical vibrations. As shown in FIG. 6, the vibration actuator 412 is located along a mid-section of the insole 402. In other embodiments, the vibration actuator 412 may be located at the front region 506 of the insole 402. It should be appreciated that the vibration actuator 412 is located away from the pressure sensors 404 and 406 to prevent the vibration from being sensed and inadvertently causing a false toe-to-toe or heel-to-toe gait determination.

Returning to FIG. 4, the example system 400 also includes the processor 408 and the memory device 410 to determine whether the vibration actuator 412 is to be activated based on the first pressure data 420, the second pressure data 422, and/or the data 424. The example processor 408 is communicatively coupled to the pressure sensors 404 and 406 and the inertial measurement unit 414 via a wired or wireless connection. The processor 408 is communicatively coupled to the memory device 410 via a wired connection. In some embodiments, the memory device 410 is integrated with the processor 408.

The processor 408 may include any microcontroller, controller, logic device, application specific integrated circuit (“ASIC”), server, workstation, etc. The memory device 410 may include any persistent or temporary memory including random access memory (“RAM”), read only memory (“ROM”), flash memory, magnetic or optical disks, optical memory, or other storage media. The memory device 410 stores one or more instructions that define an algorithm or software application 426. Execution of the instructions by the processor 408 cause the processor 408 to perform the operations discussed herein. This includes the determination of a current gait pattern among a heel-to-toe gait pattern and a toe-to-toe gait pattern for a patient.

The example processor 408 uses the data 420, 422, and/or 424 to determine a likely gait pattern of a patient. If a toe-to-toe gait pattern is detected, the processor 408 is configured to transmit an actuation signal 427 to the vibration actuator 412, which causes a haptic feedback to be communicated to the patient. The haptic feedback is indicative that the patient is walking in a toe-to-toe gait and should attempt to walk in a heel-to-toe gait. In some instances, the processor 408 is configured to transmit the actual signal 427 if a consecutive number of toe-to-toe steps are detected. In an example, the consecutive number of toe-to-toe steps may be between two and ten steps, preferably between three and five steps.

In addition to providing detecting a patient's gait and providing haptic feedback, the example processor 408 may also create a log of a patient's steps. For instance, the processor 408 may store to a log file 428 in the memory device 410 information that is indicative of a number of detected toe-to-toe steps, heel-to-toe steps, and/or a number of instances where haptic feedback was provided. In some embodiments, the processor 408 may provide a timestamp for each detected step, an indication as to whether the step was a heel-to-toe gait or a toe-to-toe gait, and an indication when the haptic feedback was provided. The use of timestamps enables a child's walking patterns to be reconstructed. For example, a clinician may use the patterns to determine if haptic feedback is effective in correcting a toe-to-toe gait.

The example system 400 may include a transceiver 430 communicatively coupled to the processor 408 to enable access to the log file 428. The transceiver 430 may include a USB transceiver, a Bluetooth® transceiver, a Zigbee® transceiver, a Wi-Fi transceiver, etc. The transceiver 430 may be directly connected to a server 432 (e.g., a workstation) and/or connected to the server 432 via a network 434 (e.g., the Internet). A clinician may use the server 432 to read the log file 428 from the memory device 410 to determine how a gait treatment is progressing. The transceiver 430 may transmit contents of the log file 428 in real time or near real time to the server 432.

In some embodiments, the clinician may use the server 432 to change parameters of a treatment that are stored in the memory device 410. The parameters may define a haptic feedback duration, a haptic feedback amplitude/frequency, a consecutive step threshold for trigging haptic feedback, pressure sensor force thresholds, etc. In some embodiments, the server 432 may be used to calibrate the pressure sensors 404 and 406 and/or the inertial measurement unit 414.

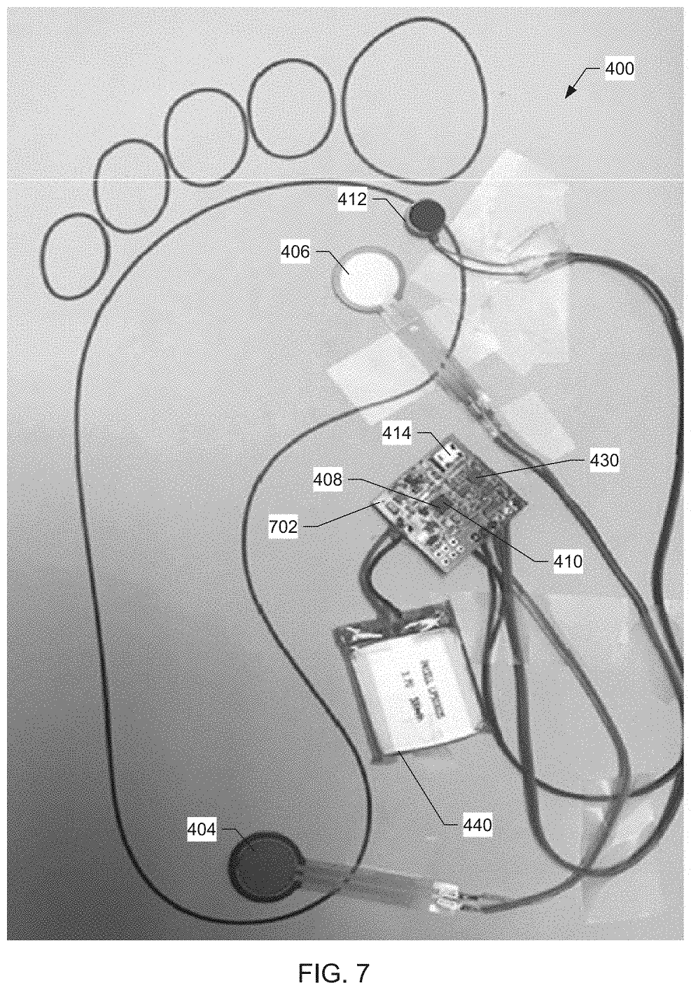

FIG. 7 shows the system 400 of FIGS. 4 to 6 connected to a representation of a child's foot via the insole 402, according to an example embodiment of the present disclosure. In the illustrated example, the processor 408, the memory device 410, the inertial measurement unit 414, and the transceiver 430 are located on a circuit board 702. The first pressure sensor 404 is placed within the insole 402 to be aligned with a heel of a patient and the second pressure sensor 406 is placed within the insole 402 to be aligned with a front-portion of a patient's foot (e.g., under a patient's toes). The vibration actuator 412 in this embodiment is also placed within the insole 402 to be aligned with a front-portion of a patient's foot.

As shown in FIG. 6, the circuit board 702 may be sandwiched between the insole 402 and the arch support 502. In other embodiments, the circuit board 702 may be positioned inside the insole 402 and aligned with a patient's arch. A power source 440 (such as a battery) may also be located between the insole 402 and the arch support 502 or positioned inside the insole 402. In other embodiments, the power source 440 and the circuit board 702 may be located in a housing that is connectable to an outside of a patient's shoe or ankle.

The example power source 440 may include any battery including a lithium-ion battery. In some embodiments, the power source 440 may include an interface for connection to an electrical outlet. Further in some embodiments, the power source 440 may include one or more transducers for converting movement into stored electrical energy.

### Example Method to Treat ITW

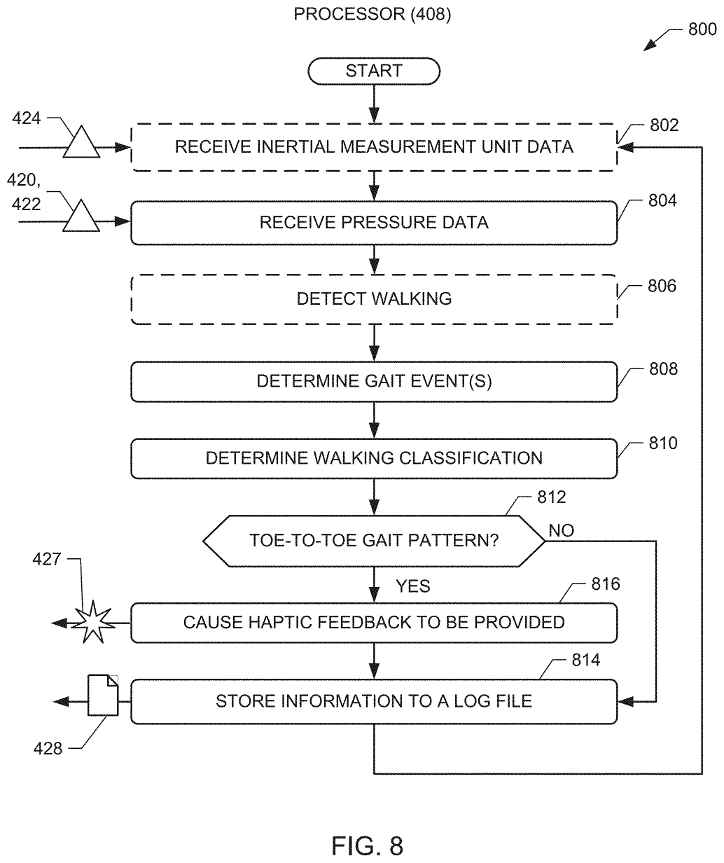

As disclosed above, the example memory device 410 stores instructions defining a software application and/or algorithm 426 for detecting a child patient has a toe-to-toe gait. FIG. 8 is a flow diagram of an example procedure 800 for detecting a child patient has a toe-to-toe gait, according to an example embodiment of the present disclosure. Although the procedure 800 is described with reference to the flow diagram illustrated in FIG. 8, it should be appreciated that many other methods of performing the steps associated with the procedure 800 may be used. For example, the order of many of the blocks may be changed, certain blocks may be combined with other blocks, and many of the blocks described may be optional. In an embodiment, the number of blocks may be changed. For instance, steps related to the inertial measurement unit may be omitted. The actions described in the procedure 800 are specified by one or more instructions and may be performed among multiple devices including, for example, the processor 408 and/or the server 432 of FIG. 4.

The example procedure 800 begins when the processor 408 receives inertial measurement unit data 424 from the inertial measurement unit 414 (block 802). The data 424 is indicative of lateral and/or angular acceleration of a foot of a patient. In instances where the system 400 of FIG. 4 omits the inertial measurement unit 414, the procedure 800 may omit this step.

As shown in FIG. 8, the example processor 408 next receives the pressure data 420 and 422 from the respective pressure sensors 404 and 406 (block 804). In some embodiments, the processor 408 may transmit a signal to each of the pressure sensors 404 and 406 to trigger the transmission of a pressure measurement.

In some embodiments, the processor 408 is configured to determine if a patient is walking (block 806). The determination may be made based on the data 424 indicative of lateral and/or angular acceleration. For example, acceleration data indicative of movement between 0.5 feet/second and 2 feet per second may be indicative of walking while movement greater than 2 feet per second may be indicative of running. Additionally or alternatively, the processor 408 may determine if a time duration between heel and/or toe contacts at the pressure sensors 404 and 406 are indicative of movement. For example, a time duration of 0.3 second and 1.5 seconds between toe and/or heel contacts may be indicative of walking. If the data 420 to 424 indicates that a patient is resting or running, the processor 408 may pause the procedure 800 until walking is detected.

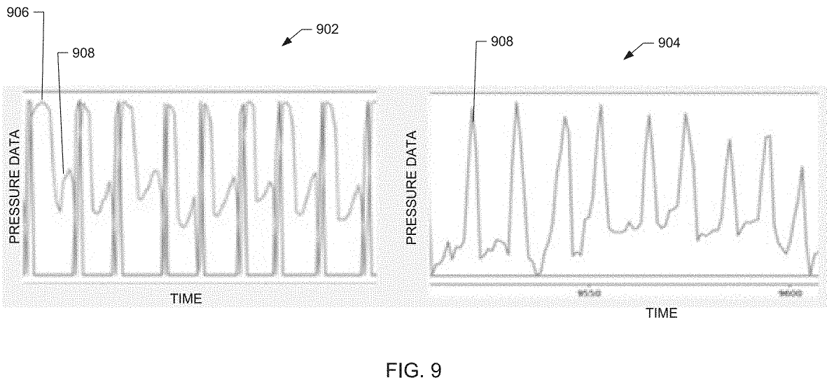

The example procedure 800 next continues for a walking patient. The processor 408 uses the data 420 to 424 to detect gait event(s) (block 808). A gait event may include a heel contact, a toe contact, a heel off, and/or a toe off. FIG. 9 is a diagram of pressure data 420 and 422 analyzed by the processor 408 to determine a gait event, according to an example embodiment of the present disclosure. FIG. 9 shows a heel-to-toe gait patent 902 and a toe-to-toe gait pattern 904. The pressure data 420 and 422 for the heel-to-toe gait pattern 902 shows a pressure spike 906 (measured by the first pressure sensor 404 at a first time) followed by a second pressure spike 908 (lower in magnitude and measured by the second pressure sensor 406 at a second, later time). The first pressure spike 906 is indicative of a heel strike and the second pressure spike 908 is indicative of a toe strike. The pressure decreases from the pressure spikes are indicative respective of the heel off and toe off events. The toe-to-toe gait pattern 904 shows only toe contact and too off events.

In some embodiments, the inertial measurement data 424 may be used to determine the events. For example, certain combinations of lateral and rotational acceleration (e.g., rotation and movement of a front foot upward) may correspond to a heel contact event while other lateral and rotational acceleration may correspond to a toe contact event (e.g., rotation and movement of a foot downward). In an embodiment, the inertial measurement data 424 may provide validation of the events determined by the processor 408 using the pressure data 404 and 406.

Returning to FIG. 8, the example processor 408 next determines a walking classification (block 810). The determination includes analyzing the walking events identified above to determine if a patient has a toe-to-toe gait pattern or a heel-to-toe gait pattern. In some embodiments, the processor 408 may combine the pressure data 420 and 422 into a single waveform to form the patterns 902 and 904, as shown in FIG. 9. To combine the data 420 and 422, the processor 408 adds together the pressure data 420 and 422 for the same time periods to determine a cumulative pressure change over time. The processor 408 may also include inertial measurement unit data 424 in the analysis.

In some instances, the processor 408 selects the first and second pressure data 420 and 422 corresponding to a same step. For example, the processor 408 may select the second data 422 among a stream of second data that is received within a time threshold of the first data 420. In other words, after detecting a heel contact event, the processor 408 determines which toe events occurred within a threshold of time, where the time threshold is between 0.2 seconds and 2 seconds. This selection ensures the heel and toe contact events are associated with the same step. In another example, the processor 408 may select the first data 420 among a buffered stream of first data that is received within a time threshold of the second data 422. This time threshold may be between 0.2 seconds and 2 seconds.

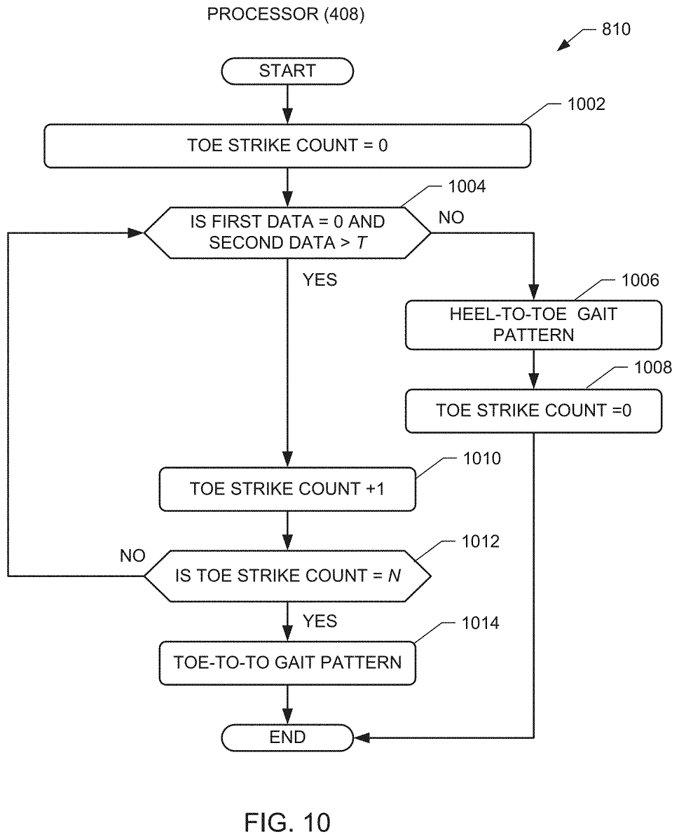

FIG. 10 is an example routine that may be performed by the processor 408 to perform the walking classification of block 810 using the one or more detected gait event(s), according to an example embodiment of the present disclosure. The example processor 408 performs the classification by first setting a toe strike count variable to zero (block 1002). The processor 408 then determines if the first data 420 (related to a heel strike) is equal to 0 and the second data 422 is above a threshold T (block 1004). In some embodiments, the processor 408 may also determine if the first data is less than a threshold TH that is indicative of an absence of a heel strike. The threshold TH may be set based on a weight of a patient and be between 0.25 lb/in² and 0.75 lb/in². The threshold N may be set based on a weight of a patient and be between 0.5 lb/in² and 1.25 lb/in².

If the conditions in block 1004 are not satisfied, the processor 408 determines the patient has a current heel-to-toe gait pattern (block 1006). The processor 408 then resets the toe strike count variable to zero (block 1008) and returns to the procedure 800 of FIG. 8. As shown in FIG. 8, if the heel-to-toe gait pattern is detected (block 812), the processor 408 stores information indicative of the heel-to-toe gait pattern to the log file 428 (block 814). The information may include a timestamp. The processor 408 then returns to block 802 and/or 804 for the next data 420 to 424.

Returning to FIG. 10, if the conditions of block 1004 are satisfied, the processor 408 increments the toe strike count variable by one (block 1010). The processor 408 then determines if a value of the toe strike count variable is equal to a threshold N (block 1012). If the threshold N has been reached, the processor 408 determines that the patient has a toe-to-toe gait pattern (block 1014). The threshold N may be between one and ten, preferably between three and five.

Returning to FIG. 8, the processor 408 determines the patient has a toe-to-toe gait pattern (block 812) and causes the vibration actuator 412 to provide haptic feedback 427 (block 816). For example, if the processor 408 detects a toe-to-toe gait pattern, the processor 408 causes the vibration actuator 412 to provide a one-second vibration. The haptic feedback provides a cue to the patient to bear their weight through their heel when walking. This haptic vibration is only detected by the patient. The processor 408 repeats the procedure 800 to provide a one-second haptic feedback every, for example, three toe strikes (e.g., toe strikes #3, #6, and #9). If the processor 408 detects ten consecutive toe strikes (e.g., toe-to-toe gait patterns), a 30-second vibration feedback is applied, which can be terminated with the patient providing weight bearing through their heel. The processor 408 stores information to the log file 428 that is indicative of the detected toe-to-toe gait pattern and/or the haptic feedback applied (block 814). The processor 408 then returns to block 802 and/or 804 for the next data 420 to 424.

Again returning to FIG. 8, if the condition in block 1012 is not satisfied, the processor 408 returns to block 1004 when additional data 420 to 424 is received during the procedure 800 of FIG. 8. The processor 408 continues progressing through the conditions in blocks 1004 and 1012 until either the toe-to-toe or the heel-to-toe gait pattern is detected. The example processor 408 continues through the procedure 800 a number of times until the procedure 800 is paused (or ended) after detecting the patient is no longer walking or a treatment session is terminated.

In some embodiments, the disclosed system 400 not only attempts to differentiate between TW versus heel-to-toe walking, but also provides a multi-day evaluation of gait characteristics. The multi-day evaluation provides physical therapists with vital information on performance of daily activities and energy expenditure in children with ITW. The goal of the example system 400 disclosed herein is to increase the frequency of a heel-toe gait pattern in children. The disclosed system also rewires neural circuits of children to help them continuously walk normally.

Long-term potential benefits for children with ITW include improved function and physical activity over their lifetime with decreased financial strain and emotional stress. Long term benefits for physical therapists include improved ability to detect changes to the TW gait pattern in the natural environment. Successful development of the feedback training using the disclosed system 400 could also be included in practice guidelines as an option for intervention with ITW.

### Other Diagnostic Usages

The example system 400 was discussed above in connection with treating children diagnosed with ITW. It should be appreciated that the system 400 may be used for other treatments including autism, developmental and coordination disorders, and cerebral palsy. For autism, a patient may be provided additional training time. Further, treatment parameters may be modified with an appropriate threshold N before haptic feedback is provided.

For developmental and coordination disorders, the example system 400 may be used in rehabilitation for children with coordination issues. In these uses, feedback parameters may be adjusted to provide feedback at a slower place based on rehabilitation milestones. For example, the threshold N may initially be set between 10 or 20 steps and then overtime reduced to two or 3 steps before feedback is provided. Further, depending on nerve sensitivity, the amplitude of the feedback may be increased.

For cerebral palsy, the example system 400 may be used in rehabilitation. Further, the example system 400 may be used in conjunction with electrical stimulation to assist with muscle activation. In these uses, feedback parameters may be adjusted to provide feedback at a slower place based on rehabilitation milestones. For example, the threshold N may initially be set between 10 or 20 steps and then overtime reduced to two or 3 steps before feedback is provided. Further, depending on nerve sensitivity, the amplitude of the feedback may be increased.

### CONCLUSION

Unless otherwise indicated, all numbers expressing quantities of ingredients, properties such as molecular weight, reaction conditions, and so forth used in the specification and claims are to be understood as being modified in all instances by the term “about.” As used herein the terms “about” and “approximately” means within 10 to 15%, preferably within 5 to 10%. Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The invention is claimed as follows:

1. A wearable apparatus for treating a child patient with Idiopathic Toe Walking (“ITW”), the apparatus comprising:
a wearable shoe insole;
a first pressure sensor located at a heel region of the shoe insole;
a second pressure sensor located at a front region of the shoe insole;
at least one vibration actuator included within the shoe insole;
a processor communicatively coupled to the first pressure sensor, the second pressure sensor, and the at least one vibration actuator; and
a memory device communicatively coupled to the processor, the memory device storing one or more instructions that define an algorithm to determine a gait pattern among a heel-to-toe gait pattern and a toe-to-toe gait pattern, execution of the one or more instructions by the processor causing the processor to:
receive first data from the first pressure sensor,
receive second data from the second pressure sensor,
determine the gait pattern as the heel-to-toe gait pattern or the toe-to-toe gait pattern based on the first data and the second data, and
indicative of determining the gait pattern is the toe-to-toe gait pattern, causing the at least one vibration actuator to provide haptic feedback.

2. The apparatus of claim 1, further comprising at least one inertial measurement unit included within the shoe insole,
wherein execution of the one or more instructions by the processor further causes the processor to:
receive third data from the at least one inertial measurement unit, and
determine the gait pattern as the heel-to-toe gait pattern or the toe-to-toe gait pattern based on the first data and the second data in conjunction with the third data.

3. The apparatus of claim 2, wherein the at least one inertial measurement unit includes at least one of an accelerometer and a gyroscope.

4. The apparatus of claim 2, wherein the third data is indicative of walking and is configured to trigger the processor to select the first data and the second data.

5. The apparatus of claim 1, wherein the algorithm is configured to provide for determination of the toe-to-toe gait pattern if the first data is at least one of equal to a value of zero or less than a threshold that is indicative of an absence of a heel strike.

6. The apparatus of claim 5, wherein the algorithm is configured to provide for determination of the heel-to-toe gait pattern if the first data is at least one of greater than a value of zero or greater than the threshold that is indicative of an absence of a heel strike.

7. The apparatus of claim 1, wherein execution of the one or more instructions by the processor further causes the processor to:
count a number of subsequent toe-to-toe gait patterns without detection of a heel-to-toe gait pattern; and
after the count of the number of subsequent toe-to-toe gait patterns has reached a threshold N, cause the at least one vibration actuator to provide the haptic feedback.

8. The apparatus of claim 7, wherein execution of the one or more instructions by the processor further causes the processor to store to the memory device in a log file the number of subsequent toe-to-toe gait patterns and an indication of providing the haptic feedback.

9. The apparatus of claim 7, wherein the threshold N is between two and ten.

10. The apparatus of claim 1, wherein the haptic feedback includes a vibration between 0.5 seconds and two seconds.

11. The apparatus of claim 1, wherein execution of the one or more instructions by the processor further causes the processor to select the second data among a stream of second data that is received within a time threshold of the first data.

12. The apparatus of claim 11, wherein the time threshold is between 0.2 seconds and 2 seconds.

13. The apparatus of claim 1, wherein execution of the one or more instructions by the processor further causes the processor to select the first data among a buffered stream of first data that is received within a time threshold of the second data.

14. The apparatus of claim 13, wherein the time threshold is between 0.2 seconds and 2 seconds.

15. The apparatus of claim 1, further comprising a battery to provide power to the processor, the memory device, the first pressure sensor, the second pressure sensor, and the at least one vibration actuator.

16. The apparatus of claim 1, wherein the front region includes a region aligned with a first metatarsal, a second metatarsal, a third metatarsal, a first phalange, a second phalange, or a third phalange of the child patient.

17. The apparatus of claim 1, wherein the vibration actuator is located along a mid-section of the shoe insole.

18. The apparatus of claim 1, wherein the vibration actuator is located at the front region of the shoe insole.

19. The apparatus of claim 1, wherein execution of the one or more instructions by the processor further causes the processor to combine the first data and the second data for determination of the gait pattern.

20. A wearable method for treating a child patient with Idiopathic Toe Walking (“ITW”), the method comprising:
receiving, in a processor, first data from a first pressure sensor located at a heel region of a shoe insole;
receiving, in the processor, second data from a second pressure sensor located at a front region of the shoe insole;
determining, via the processor, heel contact events and heel off events based at least on the first data;
determining, via the processor, toe contact events and toe off events based at least on the second data;
determining, via the processor, a heel-to-toe gait pattern if at least one heel contact event or heel off event is detected;
determining, via the processor, a toe-to-toe gait pattern if heel contact events or heel off events are not detected; and
after determining the toe-to-toe gait pattern, causing, via the processor, at least one vibration actuator included within the shoe insole to provide haptic feedback.

21. The method of claim 20, further comprising:
counting, via the processor, a number of consecutive toe-to-toe gait patterns without detection of the heel-to-toe gait pattern; and
after the count of the number of subsequent toe-to-toe gait patterns has reached a threshold N, causing, via the processor, the at least one vibration actuator to provide the haptic feedback.

22. The method of claim 21, wherein the threshold N is between two and ten.

23. The method of claim 20, further comprising:
receiving, in the processor, third data from the at least one inertial measurement unit; and
determining at least one of the heel contact events, the heel off events, the toe contact events, or the toe off events using additionally the third data.

24. The method of claim 20, further comprising:
receiving, in the processor, third data from the at least one inertial measurement unit; and
determining the third data corresponds to walking; and
after determining the child patient is walking, selecting, via the processor, the received first data and the received second data for determining the heel contact events, the heel off events, the toe contact events, and the toe off events.
